(19) United States
(12) Patent Application Publication
(10) Pub. No.: US 2026/0256432 A1
(43) Pub. Date: Sep. 3, 2026

(54) ADJUSTABLE FOOT FOR A MEDICAL IMAGING DEVICE, IN PARTICULAR FOR A COMPUTED TOMOGRAPHY DEVICE

(71) Applicant: Siemens Healthineers AG, Forchheim (DE)

(72) Inventors: Florian DIETRICH, Forchheim (DE); Rita KRUG, Fuerth (DE)

(73) Assignee: Siemens Healthineers AG, Forchheim (DE)

(21) Appl. No.: 19/552,517

(22) Filed: Feb. 27, 2026

(30) Foreign Application Priority Data
Feb. 28, 2025 (DE) 10 2025 107 653.7

(51) Int. Cl. A61B 6/03

(52) U.S. Cl. CPC A61B 6/035

(57) ABSTRACT

An adjustable foot for a medical imaging device, comprises: an elastic element configured to elastically spring-mount the device relative to the substrate; and a tensioning apparatus including a rigid element, wherein the rigid element is configured and arranged in the adjustable foot such that, on placement of the medical imaging device, said rigid element is arranged between the elastic element and the substrate. The tensioning apparatus is configured to tension the elastic element variably with the aid of the rigid element to set a spring effect of the elastic element.

### CROSS-REFERENCE TO RELATED APPLICATION(S)

The present application claims priority under 35 U.S.C. § 119 to German Patent Application No. 10 2025 107 653.7, filed Feb. 28, 2025, the entire contents of which are incorporated herein by reference.

### FIELD

One or more example embodiments of the present invention relate to an adjustable foot for a medical imaging device, a medical imaging device, in particular a computed tomography device and a method for adjusting a medical imaging device, in particular a computed tomography device.

### BACKGROUND

The components installed into systems with computed tomography devices are normally subject to production variations which can influence, inter alia, the weight and the center of gravity of these components. Since, during operation, the gantry of the computed tomography device rotates with a high speed and energy, it is normally necessary to balance these systems. For balancing, for example, the movement of the device relative to the floor is acquired with sensors and evaluated. For this purpose, a placement of the system that is flexible is helpful in order to be able to check vibrations with suitable clarity. For balancing and also for decoupling the device from the substrate, for example, damper elements are used in adjustable feet of the device. Such adjustable feet are disclosed, for example, in DE 10 2014 214 513 A1. In DE 10 2014 214 513 A1, a possibility is described for screwing such adjustable feet firmly to the floor, in particular, for earthquake-proofing, and at the same time, to be able to compensate for unevenness in the substrate.

On the other hand, it is desirable that the computed tomography device is sufficiently stable in operation, that is, in particular, it undergoes as little vibration as possible. Due to the ever-improving verification regarding structural safety and through the setting of higher standards for the design of the systems, so much material can now often be saved that the inherent frequency of the device in newer computed tomography devices is very close to the excitation frequency due to the rotation of the gantry. Under some circumstances, this can lead to visible vibrations during operation.

The requirement for balancing the system sufficiently and, at the same time, for preventing severe vibrations during operation therefore often limits the possibilities for designing the system to be lighter and to save material. In the prior art, therefore, previously the structure of the gantry has typically been so rigid and provided with so much material that the effect of resonance amplification does not arise on adjustable feet with corresponding damper elements. Alternatively, in systems that rotate relatively slowly in operation, balancing can be dispensed with. It is, however, desirable, even for lighter systems, to enable balancing in the simplest possible manner without having to accept excessive vibration during operation.

### SUMMARY

It is an object of one or more example embodiments of the present invention to enable balancing of computed tomography devices, wherein an operation of the computed tomography devices should be impaired as little as possible, in particular, by vibration.

At least this object is achieved by way of an adjustable foot, a medical imaging device and a method as claimed in the independent claims. Further features and advantages are disclosed in the dependent claims, the description and the attached drawings.

Independent of the grammatical term usage, individuals with male, female or other gender identities are included within the term.

In the following, the solutions according to one or more example embodiments of the present invention are described in relation to the claimed method and the claimed computed tomography device, the claimed adjustable foot and in relation to the other aspects of one or more example embodiments of the present invention. Features, advantages or alternatives and embodiments that are able to be combined and are described in relation to one of the aspects can be attributed to the respective other aspects of embodiments of the present invention and vice versa. For example, claims and embodiments of the method can be improved with features that are described or claimed in the context of the adjustable foot or the computed tomography device, and vice versa. Functional features of the method are implemented by way of physical units or apparatuses of the adjustable foot and/or the computed tomography device.

According to a first aspect of one or more example embodiments of the present invention, an adjustable foot is provided for a medical imaging device, in particular, a computed tomography device, for placing the medical imaging device on a substrate. The adjustable foot comprises the following: an elastic element for elastically spring-mounting the device relative to the substrate and a tensioning apparatus comprising a rigid element. The rigid element is configured and arranged in the adjustable foot such that, on placement of the medical imaging device, said rigid element is arranged between the elastic element and the substrate. The tensioning apparatus is configured to tension the elastic element variably with the aid of the rigid element and thereby to set a spring effect of the elastic element. Advantageously, with the adjustable foot, according to one or more example embodiments of the present invention, an elastic behavior for balancing can be enabled as required and, at the same time, this elastic behavior can be eliminated or lessened and/or damped with the tensioning apparatus, in particular, during operation of the medical imaging device and/or the computed tomography device.

The adjustable foot is configured for a medical imaging device. This means that the adjustable foot is configured to support the medical imaging device, possibly together with further adjustable feet, preferably also configured according to one or more example embodiments of the present invention. In particular, it can be provided that the medical imaging device can be supported by a plurality of adjustable feet, for example, three to eight, preferably four or five, particularly preferably four. The adjustable foot can be configured, for example, with regard to its carrying capacity, to support the imaging device with a suitable number of further adjustable feet. The adjustable foot according to one or more example embodiments of the present invention is particularly well configured for the requirements of a computed tomography device. With a computed tomography device, in particular, the initial balancing before putting it into operation combined with the stable base during operation is advantageous, in particular, if a rotating gantry is used. However, the adjustable foot can also be generally used for other imaging devices, particularly in cases where they are to be balanced before being put into service. The adjustable foot is configured, in particular, to be fastened on the medical imaging device, in particular, on a housing of the medical imaging device and/or the computed tomography device. Preferably, the adjustable foot is height-adjustable. In particular, the adjustable foot is height-adjustable in such a manner that with the adjustable foot, in particular, with a plurality of adjustable feet, the medical imaging device can be oriented with regard to its position. The adjustable foot is configured so that the imaging system can be placed, via the adjustable foot, on a substrate. The substrate can be, in particular, a ground and/or a floor.

The adjustable foot comprises an elastic element for elastically spring-mounting the system relative to the substrate. The elastic element can also be designated a damper element and/or an elastic damper element. The elastic element can be configured in one piece. The elastic element can be, for example, an elastic cushion element and/or an elastic ring element. The geometric configuration of the elastic element and the geometric configuration of the rigid element can be, for example, adapted to one another. For example, the elastic element can comprise at least one recess and the rigid element can comprise a corresponding projection and/or shoulder, or vice versa. The elastic element can be configured, optionally in cooperation with the tensioning apparatus and/or the rigid element, to decouple the imaging device from the substrate vibrationally. With the elastic element, a certain degree of freedom of movement in a plane parallel to the substrate and perpendicular thereto is enabled, by which a balancing of the imaging device is improved, or even enabled at all.

The adjustable foot further comprises a tensioning apparatus with a rigid element. The rigid element can be configured in one piece. In the state of the adjustable foot and/or the imaging device placed as intended with the adjustable foot, the rigid element can have a greater extent parallel to the substrate than perpendicularly thereto. An extent perpendicularly to the substrate preferably corresponds substantially to the direction along which the elastic element and the rigid element are sequentially arranged. The rigid element is more rigid than the elastic element. The rigid element has, in particular, a larger modulus of elasticity than the elastic element and/or is less elastic than the elastic element. When the imaging device is installed, the rigid element is arranged between the elastic element and the substrate. In particular, the rigid element is configured to be in contact with the substrate and/or to stand on the substrate. Preferably, an underside of the rigid element can be configured to be slip-proof, in particular, slip-proof relative to a substrate on which the medical imaging device, in particular, the computed tomography device is typically placed. The underside of the rigid element can be understood, in particular, to be a side of the rigid element facing away from the elastic element. For example, the underside of the rigid element can be configured to have an adhesive force on the substrate that, in particular, counteracts and/or prevents a shifting of the imaging device.

The tensioning apparatus is configured to tension the elastic element variably with the aid of the rigid element and thereby to set a spring effect of the elastic element, in particular for the adjustable foot. A setting can be understood in this case to be an adjustment. The setting can comprise, in particular, a release of the elastic element so that the elastic element is not substantially impaired in its elasticity by the rigid element, and a complete or approximately complete negation of the spring effect of the elastic element. In particular, the tensioning apparatus can be configured so that in the process of a setting by tensioning and/or clamping with the rigid element, the elastic element is effectively completely deprived of its spring effect and, in a further setting by relaxing the tensioning apparatus, its full spring effect can develop. Advantageously, therefore, the functionality of the elastic element can be substantially completely activated, and in a further setting, reduced or even substantially eliminated. Preferably, the tensioning apparatus is configured to cause a tensioning of the elastic element in its completely or approximately completely reversible range. With the tensioning apparatus, a variably settable tensioning of the elastic element and therefore, in particular, a variable damping can also be achieved in a targeted and settable manner. By way of the tensioning apparatus, a balancing of the imaging device can advantageously also be enabled during retrofitting, converting and/or servicing the imaging device, for example, in the form of a component exchange, by simple loosening of the tensioning apparatus, after which, through renewed tensioning with the tensioning apparatus, a normal operation with little or no influence by the elastic element can be restored relatively easily. In particular, as compared with a tensioning against the substrate, the tensioning with the rigid element offers further advantages. In particular, the tensioning of the imaging device can thus be carried out independently of the selected substrate. The functionality of the adjustable foot is therefore not dependent upon the placement site of the imaging device and/or upon the nature of the respective substrate. In the context of this invention, it has been recognized that the substrates used for medical imaging devices, in particular, also for computed tomography devices, often only have a limited rigidity and hardness. Thus, through the use of the rigid element, a bridging of the elasticity of the elastic element can be enabled better, more reliably and independently of the substrate. Optionally, the rigid element can also enable an improved stability and/or a more predictable standing of the imaging device.

According to one embodiment, the tensioning apparatus comprises a fixing element, in particular a screw and/or a clamp for variable tensioning of the elastic element relative to the rigid element. For example, the tensioning apparatus can comprise a quick clamping mechanism, in particular with a clamp. The quick clamping mechanism can be based, for example, upon a mechanism corresponding to a toggle clamp or a knee-lever clamp. A particularly simple solution can be a fixing element in the form of a screw. The screw can be arranged or able to be arranged substantially centrally in the adjustable foot. The tensioning apparatus can be configured to pull the elastic element against the rigid element with the aid of the screw by screwing them together. For tensioning, the screw can comprise a screw head. The screw can comprise a screw head drive, in particular, in and/or on the screw head. For example, the screw head drive can be a slot head, a polygonal socket, in particular a hexagonal socket or a cross-head. Optionally, a plurality of fixing elements, for example, different fixing elements or a plurality of identical fixing elements, for example, a plurality of screws and/or a plurality of clamps can be provided.

According to one embodiment, the tensioning apparatus comprises a screw as the fixing element for variable tensioning of the elastic element against the rigid element, wherein the rigid element comprises an upper opening with an internal thread for screwing in of the screw and/or wherein the rigid element comprises a through opening which is configured so that the screw can be fed through the through opening, in particular, for screwing the screw into the substrate. The tensioning apparatus can be configured so that on screwing to the substrate, the elastic element is pulled against the rigid element, in particular, with a seating element that, by way of the screwing, presses the elastic element against the rigid element. Alternatively, this effect can be achieved still better in that the screw is screwed directly into the rigid element with the aid of the internal thread of the rigid element and thus the rigid element is pulled directly toward the elastic element. In both cases, it can be provided that the elastic element is tensioned or able to be tensioned between the rigid element and the seating element. A single screw can be provided for variable tensioning or a plurality of screws can be provided for variable tensioning. The screw can be, in particular, a central screw that is arranged substantially centrally in the adjustable foot. A central screw can be a possibility for realizing an even tensioning in an effective manner with only one single screw. Advantageously, therefore, the material expenditure and also the effort involved in screw fastening are reduced. The opening of the rigid element can preferably be arranged in the middle and/or centrally in the rigid element, in particular, if a central screw is provided. The rigid element can be configured annular. The opening of the rigid element can have a variable diameter. For example, an upper region of the rigid element can have a larger diameter here for the passing through of a sleeve, in particular, a sleeve as described here, with the screw and a smaller lower diameter for passing through and/or screwing the screw. The lower diameter can comprise the internal thread for screwing the screw. The elastic element can comprise a feedthrough, in particular, a central feedthrough and the tensioning apparatus can be configured so that the screw is fed through the feedthrough. The feedthrough of the elastic element can have a larger diameter on a side of the elastic element facing toward the rigid element than on the side facing away. The larger diameter can be provided for receiving a projection of the rigid element. The rigid element can have a projection and/or shoulder, in particular around its opening, in particular a projection extending peripherally around the opening which, in particular, is adapted to the larger diameter of the elastic element. The peripherally extending projection can be configured so that between the screw, in particular, the central screw, and the projection, at least one portion of a sleeve, in particular, a sleeve as described herein can additionally be arranged, wherein the screw is or can be guided through the sleeve. With a projection of this type, a stiffening of the adjustable foot can be better controlled and, in particular, vibrations in the transverse direction can be better restricted. Preferably, the projection of the rigid element can be spaced from the elastic element upwardly and/or in the screwing direction. Advantageously, therefore, in the relaxed state, a less rigid behavior of the elastic element can be achieved. In this way, the balancing can be better performed.

According to one embodiment, the adjustable foot comprises a seating element, in particular, a seating plate which, in the placed state of the adjustable foot, lies on the elastic element, wherein the tensioning apparatus is configured to tension the elastic element variably between the seating element and the rigid element. The seating element can function as a pressing part which presses and/or can be pressed from above onto the elastic element for tensioning. Preferably, the seating element, in particular, the seating plate is produced from a rigid material. The rigid material from which the seating element is made can correspond to the rigid material of the rigid element. The same features that are described in this application in relation to the material properties of the rigid element can also be provided for the seating element. In particular, the seating element can be produced from the same material as the rigid element. It can alternatively be provided that the seating element is made from a material that is different from the material of the rigid element. For example, both can have a modulus of elasticity that is in the range as described for the rigid element, wherein however, within this range a different modulus of elasticity is provided for the seating element and the rigid element. The seating plate can be produced, in particular, from metal, preferably steel. Preferably, the seating plate is produced so that its edge region at least partially externally encompasses the elastic element. The elastic element can have an exterior shoulder, in particular, extending circumferentially, upon which an edge region of the seating plate lies. In particular, it can be provided that the edge region of the seating plate at least partially surrounds the elastic element in an upper region of the elastic element above the shoulder and lies upon the shoulder of the elastic element.

According to one embodiment, the seating element, in particular the seating plate, comprises a through opening for the passing through of the screw. Thus, as described herein, the screw can advantageously be guided through the seating element in order to tension the elastic element with the rigid element. The through opening can be configured so that a sleeve, in particular, a sleeve as described herein can be guided with the screw through the through opening.

According to one embodiment, the adjustable foot comprises a sleeve, in particular, a threaded sleeve with an external thread for screwing to a frame of the imaging device, in particular, a computed tomography device, wherein the sleeve comprises a through opening that is configured such that the screw can be guided through the through opening. In its basic form, the sleeve can correspond to an elongate hollow cylinder. In particular, the elastic element comprises a feedthrough and the sleeve protrudes at least partially into the feedthrough. The opening of the rigid element, the through opening of the seating element, the through opening of the sleeve and the feedthrough of the elastic element can be conceptually similar or identical. In particular, they are all provided for passing through or introducing of the screw. The opening, the through openings and the feedthrough can have different diameters individually and between themselves. The through opening of the seating element and the feedthrough of the elastic element can have, for example, a somewhat larger diameter for the passing through of the sleeve. The opening of the rigid element can have a smaller diameter, at least partially, for the passing through or screwing in of the screw. In the region of the smaller diameter, the opening can have an internal thread, in particular, as described herein. The external thread can be configured such that therewith a height setting of the adjustable foot can be adjusted. The imaging device, in particular, a housing of the imaging device can comprise an internal thread complementary to the external thread for this purpose. The sleeve can comprise an actuating region which is configured such that the sleeve can be rotated with a turning tool, for example, with a torque key and/or a polygonal key such as a hexagonal key. The actuating region can be, in particular, a polygon, in particular, a hexagon. The actuating region is preferably arranged on an end of the sleeve facing away from the rigid element. In combination with the external thread of the threaded sleeve, an actuating region of this type can be a particularly efficient possibility for enabling a simple height adjustment capability for the adjustable foot. The sleeve can preferably be configured in one piece. With a configuration in one piece, a particularly good loading capacity of the sleeve and/or of the adjustable foot can be achieved.

In a lower region for introduction into an opening of the rigid element, the sleeve can have a groove, in particular, an external circumferential groove for a snap ring.

The adjustable foot can have a securing ring and/or snap ring that is arranged in the groove of the sleeve for securing the sleeve in the rigid element. The securing ring and/or snap ring can serve to secure the rigid element and the elastic element against falling off the adjustable foot, in particular, if the screw is screwed out of the rigid element.

Preferably, the sleeve can be configured to lie on the seating element as described herein. The screw and the sleeve can be configured so that a screw head presses the sleeve from above against the seating element. Since at the same time, the rigid element is tightened from below by the screw, the elastic element can thus be clamped between the seating element and the rigid element. The seating element can have, on its through opening, an upwardly widening bevel on which the sleeve or a shoulder of the sleeve as described below lies or can lie. The sleeve can also have a corresponding bevel on its seating point. By way of the bevel, a more flexible seating of the sleeve on the seating element can be enabled. In particular, an orientation of the adjustable foot can thus be adjustable.

According to one embodiment, the sleeve has at least one shoulder which rests upon the seating element so that the elastic element can be variably clamped between the seating element and the rigid element with the aid of the sleeve and the screw. In particular, it can be provided that a lower part of the sleeve is or can be introduced beneath the shoulder through the through opening of the seating element into the feedthrough of the elastic element and optionally at least partially into the opening of the rigid element. In this way, a particularly good stability of the adjustable foot can be enabled.

According to one embodiment, the adjustable foot is configured such that, also in a clamped state of the elastic element, the sleeve is spaced from the rigid element. It is thus provided that a play remains between the sleeve and the rigid element at all times. In this way, the reliability of the adjustable foot can be increased and it can be ensured that a placement of the sleeve on the rigid element prevents a tensioning of the elastic element.

According to one embodiment, the elastic material has a Shore A hardness from 20 to 90 Shore A, preferably 50 to 80 Shore A, particularly preferably 55 to 75 Shore A, more particularly preferably 60 to 70 Shore A. Alternatively or additionally, the elastic element is made from an elastomer material and/or a natural rubber material. An elastomer is particularly well suited, firstly, to withstand the compression loading acting upon the adjustable foot and, secondly, to enable good damping and spring mounting. The compression loading is made up from the weight and/or the weight component of the imaging device and the biasing from the tensioning apparatus. In principle, a material with the specified Shore A hardness is well suited to the purposes of this invention. For this, a Shore A hardness from 55 to 75 Shore A is particularly good and better still is 60 to 70 Shore A. In particular, with a Shore A hardness from 60 to 70 Shore A, a balancing of the system can be carried out particularly well, wherein a tensioning for bridging the elastic element is also very readily possible.

According to one embodiment, the rigid element is produced from metal, in particular, steel. Alternatively or additionally, the rigid element has a modulus of elasticity of at least 60 GPa (gigapascals), preferably at least 150 GPa, particularly preferably at least 190 GPa. In general, the modulus of elasticity must not be upwardly restricted. For example, a modulus of elasticity for the rigid element of up to 1000 GPa is also conceivable, provided the rigid element is sufficiently stable. A modulus of elasticity of up to 500 GPa can be especially practicable to use. A modulus of elasticity of at least 60 GPa can enable a sufficient deformability so that one or more example embodiments of the present invention can function well. For example, the rigid element can be made of aluminum or an aluminum alloy. Better still is a modulus of elasticity of at least 150 GPa. With a modulus of elasticity of at least 150 GPa, the rigid element can be configured thinner and therefore more space-saving. For example, a rigid element made of aluminum must have a greater thickness than a rigid element made of steel, so that a sufficient stability can be ensured. A modulus of elasticity of at least 190 GPa can enable a particularly efficient elimination of the damping and/or vibration properties of the elastic element.

According to one embodiment, the rigid element is a rigid plate, in particular a metal plate, preferably a steel plate. The rigid plate can be, in particular, a flat ring. The center of the ring can be the opening of the rigid element as described herein. Optionally, the flat ring can have a shoulder and/or projection, in particular, as described herein, on its side facing toward the elastic element. A rigid plate can be particularly well suited for implementing the concept according to one or more example embodiments of the present invention. A steel plate or another suitable metal plate has material properties that are particularly suitable, in particular, a particularly suitable modulus of elasticity and good stability.

A further aspect of one or more example embodiments of the present invention is a medical imaging device, in particular, a computed tomography device comprising at least one adjustable foot, preferably a plurality of adjustable feet as described herein. All the advantages and features of the adjustable foot can be transferred likewise to the imaging device and vice versa. In particular, the imaging device and/or the computed tomography device can also be regarded as an imaging system and/or as a computed tomography system with a plurality of components, including the at least one adjustable foot. Further components can be, for example, a gantry and/or a control station. The imaging device, in particular the gantry, can comprise a housing which is supported by the at least one adjustable foot. The housing can have at least one internal thread into which the sleeve of the adjustable foot, in particular, the sleeve described herein is and/or can be screwed. The imaging device can be configured such that, with the at least one adjustable foot, in particular, by way of variable screwing-in of the adjustable foot into the internal thread of the housing, a height adjustment can be made and/or a balancing can take place. For example, an adjustable foot can be provided at each of four corner points of the imaging device, in particular, the housing of the gantry.

A further aspect of one or more example embodiments of the present invention is a method for adjusting a medical imaging device, in particular, a computed tomography device as described herein. The method comprises the following steps:
releasing the elastic element of the at least one adjustable foot by way of the tensioning apparatus so that the at least one adjustable foot is spring-mounted by the elastic element;
balancing the imaging device, in particular, computed tomography device while the elastic element is released;
at least partial elimination of the spring effect of the elastic element by tensioning the elastic element with the tensioning apparatus, in particular, in order to prepare the medical imaging device, in particular, the computed tomography device, for operational use.

All the advantages and features of the adjustable foot and of the imaging device can be transferred likewise to the method and vice versa. The adjustable foot can be adjusted with the tensioning apparatus for operational use such that an inherent frequency of the imaging device is outside a rotation frequency, in particular, the rotation frequency of the gantry during operation.

All the embodiments described herein can be combined with one another if not explicitly stated otherwise.

### BRIEF DESCRIPTION OF THE DRAWINGS

Embodiments will now be described making reference to the accompanying drawings.

FIG. 1 shows an adjustable foot for a medical imaging device for placing the medical imaging device on a substrate and/or floor according to one embodiment of the present invention;

FIG. 2 shows a front view of a computed tomography device with adjustable feet according to one embodiment of the present invention; and

FIG. 3 shows a flow diagram for a method for adjusting a medical imaging device according to an embodiment of the present invention.

### DETAILED DESCRIPTION

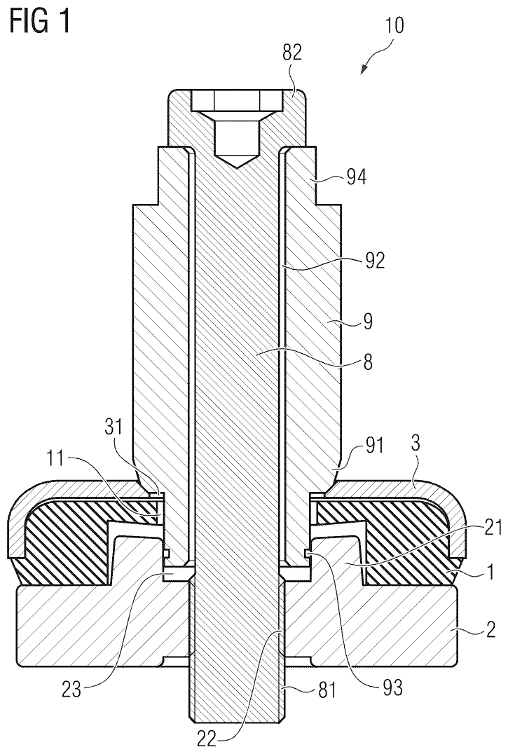

FIG. 1 shows an adjustable foot 10 for a medical imaging device, in particular, for a computed tomography device, for placing the medical imaging device on a substrate and/or floor according to one embodiment of the present invention. The adjustable foot 10 comprises an elastic element 1 for elastically spring-mounting the device relative to the substrate, and a tensioning apparatus comprising a rigid element 2 and a fixing element as well as a sleeve 9. The elastic element 1 can be made, for example, from an elastomer material or from a natural rubber. The rigid element can be produced, for example, from metal, preferably from steel. The sleeve 9 can preferably be configured as a threaded sleeve with an external thread. The adjustable foot 10 can be screwed to a frame of the imaging device with the external thread of the threaded sleeve. On its end facing away from the rigid element 2, the sleeve 9 comprises an actuating region 94 in the form of a hexagon which is configured such that the sleeve 9 can be rotated with a turning tool, for example, with a torque wrench and/or a hexagonal key.

In this embodiment, the fixing element is a screw 8 that is fed through the sleeve 9. In principle, another fixing element is also conceivable, for example, a clamp. The rigid element 2 is arranged between the elastic element 1 and the substrate. The tensioning apparatus is configured to tension the elastic element 1 variably with the aid of the rigid element 2 and the screw 8 and thereby to set a spring effect of the elastic element 1. For this purpose, the rigid element 2 has a central opening 23 with an internal thread 22 for screwing in the screw 8 with an external thread 81 of the screw 8. The opening 23 is configured as a continuous passage in this exemplary embodiment. For example, a hole can be provided in the substrate and/or floor into which the screw can be introduced under the rigid element 2. Alternatively to a screwing of the screw 8 with the internal thread 22 of the rigid element 2 as shown here, the screw 8 could also be screwed, for example, into a thread in the floor.

The adjustable foot 10 further comprises a seating element, specifically a seating plate 3 which lies on the elastic element 1. If the screw 8 is now tightened, the elastic element 1 is clamped between the seating plate 3 and the rigid element 2. Therein, the sleeve 9, through the through opening 92 of which the screw is passed, presses from above with the aid of the screw head 82 of the screw 8 against the seating plate 3 while the rigid element 2 is simultaneously tightened by the screw 8. Therein, the sleeve 9 presses with a shoulder 91 of the sleeve 9 against the seating plate 3. Thus, the elastic element 1 can be clamped between the seating plate 3 and the rigid element 2. In order that the screw 8 can be fed through the seating plate 3 and the elastic element 1 into the rigid element 2, they each have a central through opening 31 and/or a central feedthrough 11. The seating plate 3 can have, at the top on its through opening 31, an upwardly widening bevel on which the sleeve 9 and/or a shoulder 91 of the sleeve 9 rests. The shoulder 91 of the sleeve 9 also has a corresponding bevel on its seating point.

The rigid element 2 has a circumferentially extending projection 21 surrounding its opening 23. Complementary to this, the feedthrough 11 of the elastic element 1 has a larger diameter on the side of the elastic element 1 facing toward the rigid element 2 than on the side facing away therefrom. The projection 21 protrudes into the feedthrough 11 of the elastic element 1. By this mechanism, device and/or means, vibrations in the transverse direction can also be restricted better when the elastic element 1 is clamped with the rigid element. In that the projection 21 of the rigid element 2 is spaced from the elastic element 1 upwardly and/or in the screwing direction, in the relaxed state, a less stiff behavior of the elastic element 1 can be achieved since this part of the elastic element 1 does not rest upon the rigid element 2. In the relaxed state, the elastic element 1 can thus better develop its elastic effect. Below its shoulder 91, the sleeve 9 continues further through the through opening 31 of the seating plate 3 and protrudes into the feedthrough 11 of the elastic element 1 and into the opening 23 of the rigid element 2. Therefore a further portion of the sleeve 9 is arranged in the opening of the rigid element 2 and between the screw 8 and the projection 21. In this way, greater stability can be achieved. The sleeve 9 has an external circumferential groove 93 in its lower region. A securing ring and/or snap ring can be inserted into the groove 93. With the aid of the groove 93 and the securing ring and/or snap ring, the rigid element 2 and the elastic element 1 can be secured against the adjustable foot 10 falling off.

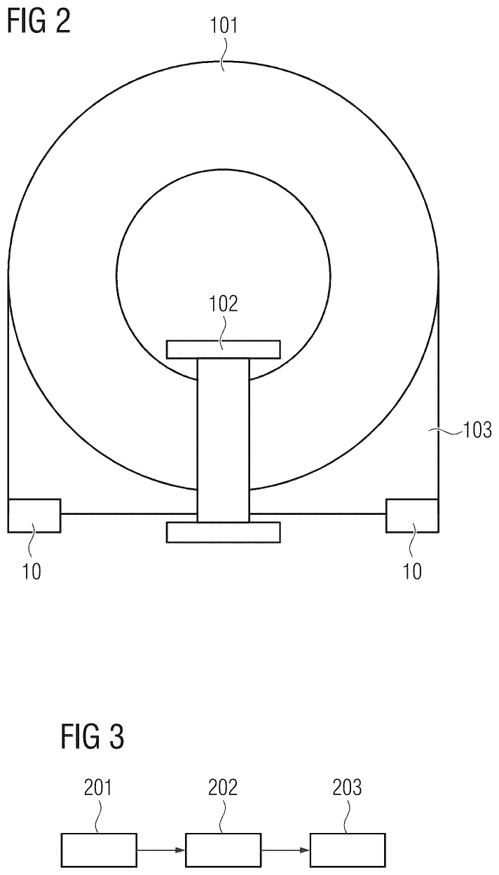

FIG. 2 shows a front view of a computed tomography device with a gantry 101 and a patient support 102 as well as an exemplary arrangement of the adjustable feet 10 according to one or more example embodiments of the present invention, which support a housing 103 of the gantry 101 according to one embodiment of the present invention. For this purpose, the adjustable feet 10 are screwed with an external thread of a sleeve 9 of the adjustable feet 10 into an internal thread in the housing 103. For example, four adjustable feet 10 can be provided at the corner regions of the housing 103, wherein the rear two adjustable feet 10 are not visible in this front view.

FIG. 3 shows a flow diagram for a method for adjusting a medical imaging device, in particular, a computed tomography device, according to an embodiment of the present invention. In a first step 201, the elastic element 1 of the at least one adjustable foot 10 is released by the tensioning apparatus so that the at least one adjustable foot 10 is spring-mounted by the elastic element 1. In a further step 202, the medical imaging device is balanced while the elastic element is released. In a further step 203, the spring effect is at least partially eliminated by way of the elastic element 1 in that the elastic element 1 is clamped with the tensioning apparatus. Thus, the imaging device is prepared for an operational use.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of the embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing embodiments. The present invention may, however, be embodied in many alternate forms and should not be construed as limited to only the embodiments set forth herein.

What is claimed is:

1. An adjustable foot for a medical imaging device, the adjustable foot configured to place the medical imaging device on a substrate, wherein the adjustable foot comprises:
an elastic element configured to elastically spring-mount the medical imaging device relative to the substrate; and
a tensioning apparatus having a rigid element, the rigid element being arranged in the adjustable foot and configured such that, on placement of the medical imaging device, said rigid element is arranged between the elastic element and the substrate, wherein
the tensioning apparatus is configured to tension the elastic element variably with the aid of the rigid element to set a spring effect of the elastic element.

2. The adjustable foot as claimed in claim 1, wherein at least one of
the tensioning apparatus includes a screw to tension the elastic element variably with the aid of the rigid element,
the rigid element has an opening with an internal thread to screw in the screw, or
the rigid element includes a first through opening to feed the screw through to screw the screw into the substrate.

3. The adjustable foot as claimed in claim 2,
wherein the adjustable foot includes a seating element, and
wherein the tensioning apparatus is configured to tension the elastic element variably between the seating element and the rigid element.

4. The adjustable foot as claimed in claim 3, wherein the seating element includes a second through opening for passing through the screw.

5. The adjustable foot as claimed in claim 3,
wherein the adjustable foot includes a sleeve, and
wherein the sleeve includes a third through opening to guide the screw.

6. The adjustable foot as claimed in claim 5,
wherein the sleeve has at least one shoulder that rests upon the seating element so that the elastic element is configured to be variably clamped between the seating element and the rigid element with the aid of the sleeve and the screw.

7. The adjustable foot as claimed in claim 1, wherein at least one of
an elastic material of the elastic element has a Shore A hardness from 20 to 90 Shore A, or
the elastic material is made from at least one of an elastomer material or a natural rubber material.

8. The adjustable foot as claimed in claim 1, wherein at least one of
the rigid element is a rigid plate, or
the rigid element has a modulus of elasticity of at least 60 GPa (gigapascals).

9. A medical imaging device, comprising:
at least one adjustable foot as claimed in claim 1.

10. A method for adjusting a medical imaging device as claimed in claim 9, the method comprising:
releasing the elastic element of the at least one adjustable foot by way of the tensioning apparatus so that the at least one adjustable foot is spring-mounted by the elastic element;
balancing the medical imaging device while the elastic element is released; and
at least partially eliminating the spring effect of the elastic element by tensioning the elastic element with the tensioning apparatus to prepare the medical imaging device for operational use.

11. The adjustable foot of claim 1, wherein the medical imaging device is a computed tomography device.

12. The adjustable foot as claimed in claim 3, wherein the seating element is a seating plate which, in a placed state of the adjustable foot, lies on the elastic element.

13. The adjustable foot as claimed in claim 5, wherein the sleeve is a threaded sleeve with an external thread for screwing to a frame of the medical imaging device.

14. The adjustable foot as claimed in claim 5, wherein the elastic element includes a feedthrough and the sleeve protrudes at least partially into the feedthrough.

15. The adjustable foot as claimed in claim 7, wherein the elastic material has a Shore A hardness from 50 to 80 Shore A.

16. The adjustable foot as claimed in claim 15, wherein the elastic material has a Shore A hardness 55 to 75 Shore A.

17. The adjustable foot as claimed in claim 16, wherein the elastic material has a Shore A hardness from 60 to 70 Shore A.

18. The adjustable foot as claimed in claim 8, wherein at least one of
the rigid plate is a metal plate,
the rigid plate is a steel plate,
the rigid element has a modulus of elasticity of at least 150 GPa, or
the rigid element has a modulus of elasticity of at least 190 GPa.

19. The adjustable foot as claimed in claim 1,
wherein the adjustable foot includes a seating element, and
wherein the tensioning apparatus is configured to tension the elastic element variably between the seating element and the rigid element.

20. The adjustable foot as claimed in claim 1,
wherein the adjustable foot includes a sleeve, and
wherein the sleeve includes a third through opening to guide a screw.
